(19) United States
(12) Patent Application Publication
(10) Pub. No.: US 2024/0148816 A1
(43) Pub. Date: May 9, 2024

(54) HERBAL COMPOSITION COMPRISING SOLANUM NIGRUM FOR THE TREATMENT OF CANCER

(71) Applicant: King Saud University, Riyadh (SA)

(72) Inventors: Haseeb Ahmad Khan, Riyadh (SA); Rajendra Prasad, Tamilnadu (IN); Amani Ahmed Alghamdi, Riyadh (SA); Salman Alrokayan, Riyadh (SA)

(73) Assignee: King Saud University, Riyadh (SA)

(21) Appl. No.: 17/981,660

(22) Filed: Nov. 7, 2022

(51) Int. Cl. A61K 36/81; A61K 45/06; A61P 35/00

(52) U.S. Cl. CPC A61K 36/81; A61K 45/06; A61P 35/00

(57) ABSTRACT

The present disclosure generally relates to herbal compositions. Specifically, the present disclosure relates to herbal composition comprising of Solanum nigrum along with a pharmaceutically acceptable excipients. The present invention also relates to a method of preparing the herbal composition comprising Solanum nigrum and its anti-proliferative effect.

### FIELD OF THE INVENTION

The present disclosure generally relates to herbal compositions. Specifically, the present disclosure relates to herbal composition comprising of Solanum nigrum and one or more pharmaceutically acceptable excipients. The present invention also relates to a method of preparing the herbal composition comprising Solanum nigrum and its anti-proliferative effect.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

The growing incidence of breast cancer and the adverse effects of anticancer chemotherapy are the major concerns to cancer researchers.

Breast cancer is a major cancer sub-type among women worldwide. The pathogenesis of breast cancer involves multiple cell types, and it is a leading cause of death among women. Breast cancer is a multi-stage process involving several endogenous and exogenous factors (Sun et al., 2017). Breast cancer accounts for 23% of all cancer deaths, particularly in post-menopausal women (Akram et al., 2017). The four main intrinsic molecular subtypes of breast cancer show differences in phenotype, prognosis, treatment response, and survival rate. The luminal A, luminal B, human epidermal growth factor receptor 2 (HER2)-enriched, and triple-negative breast cancer were the major subtypes of breast cancer (Cortazar et al., 2014; Waks & Winer, 2019). The luminal A and luminal B expresses estrogen and progesterone receptors, but not the human epidermal growth factor receptor-2 (HER-2). The HER-2-enriched breast cancer cells have only HER-2 expression, and the Triple-negative breast cancer (TNBC) shows no occurrence of any of the hormonal receptors (Carey et al., 2006).

Multimodal therapies such as surgery followed by radio-chemotherapy are the major treatment option for breast cancer. Neoadjuvant chemotherapy is a preferred treatment modality to preserve breast tissues. The severe adverse effects and toxicity in the non-target organs limit the therapeutic advantages of neoadjuvant chemotherapy (Sun et al., 2017). Therefore, there is a need for alternative and non-toxic treatment modalities for the treatment of breast cancer.

It has been proved that several medicinal plants possess specific therapeutic potential. Research studies illustrate that medicinal plants have gained potential in managing breast cancer (Guilford & Pezzuto, 2008). Solanum nigrum L. family of Solanaceae, commonly known as ‘Black nightshade,’ is a widely used medicinal plant worldwide. Solanum nigrum has been traditionally used for the prevention and treatment of various chronic diseases. It has been reported that S. nigrum leaves extract reduced oxidative stress and showed potential in quenching the formation of the radical (Campisi et al., 2019).

There is therefore a need in the art to develop an herbal composition, which is safe and effective against cancer diseases, particularly breast cancer. The present invention satisfies the existing needs, as well as others, and generally overcomes the side effects of the chemotherapy.

### OBJECTS OF THE INVENTION

Primary object of the present disclosure is to provide herbal composition comprising Solanum nigrum.

Another object of the present disclosure is to provide herbal composition comprising Solanum nigrum for the treatment of cancer.

Another object of the present disclosure is to provide herbal composition comprising Solanum nigrum for the treatment of cancer that is safe and devoid of side effects.

Other objects of the present disclosure will be apparent from the description of the invention herein below.

### SUMMARY OF THE INVENTION

The present disclosure generally relates to herbal compositions. Specifically, the present disclosure relates to herbal composition comprising of Solanum nigrum along with a pharmaceutically acceptable excipients. The present invention also relates to a method of preparing the herbal composition comprising Solanum nigrum and its anti-proliferative effect.

In an aspect, the present invention relates to a herbal composition comprising a therapeutically effective amount of an extract of Solanum nigrum and one or more pharmaceutically acceptable excipients.

In another aspect of the present invention, the extract of Solanum nigrum is an extract of leaves of Solanum nigrum, an extract of fruits of Solanum nigrum, or an extract of whole plant of Solanum nigrum.

In another aspect of the present invention, the water extract of Solanum nigrum.

In another aspect of the present invention, the extract of Solanum nigrum is present in an amount ranging from about 1% to about 99% by weight of the composition.

In another aspect of the present invention, the herbal composition is administered in combination with anticancer agents.

In another aspect of the present invention, the Solanum nigrum works as anticancer agent through eliciting immunomodulation and anti-proliferation.

In yet another aspect, the present invention relates to a method of treating a cancer by administering a therapeutically active amount of the herbal composition as claimed claim 1 to a subject in need thereof.

### BRIEF DESCRIPTION OF THE FIGURES

FIG. 1: Effect of water extract of S. nigrum (SNWE) on cell viability in MCF-7 and MDA-MB-231 cells. Cells (10,000 cells/well) were treated with increasing concentrations of SNWE ranging from 0.19-100 μg/ml for 48 h and then assessed for cell viability using MTT assay. The bar diagram represents the % of cell viability, and readings were accessed with a multimode plate reader (Molecular Devices, USA). Points with error bar represents mean±SEM.

FIG. 2: The ATP levels in the breast cancer cells were measured by colorimetric ATP assay at different time interval (24 h, 48 h, and 72 h) under the treatment of SNWE. The graph diagram represents the nM of ATP in the cells against the SNWE treatment concentrations. There was a significant decrease in the ATP levels while increase in the concentration of SNWE treatment. The data were presented as mean±SEM.

FIG. 3: SNWE on intracellular ROS generation in (a) MCF-land (b) MDA-MB-231 cells by DCFH-DA staining. Photomicrograph shows the green fluorescence under green filter (original magnification, 20×). Bar diagram represents % of fluorescence intensity made with excitation and emission at 485±10 and 530±1.5 nm, respectively, using a multimode reader (Molecular Devices, USA). Points with error bar represents mean±SEM.

FIG. 4: SNWE on the loss of mitochondrial membrane potential in both (a) MCF-7 and (b) MDA-MB-231 cells. Photomicrograph shows the fluorescence observed under a green filter (original magnification, 20×). Bar diagram represents % fluorescence intensity made with excitation and emission at 450±10 nm and 490±10 nm, respectively, using a multimode reader (Molecular Devices, USA). Points with error bar represents mean±SEM.

FIG. 5: SNWE on apoptosis in both (a) MCF-7 and (b) MDA-MB-231 cells by AO/EtBr staining. The increased concentration of SNWE showed Ao/EtBr staining due to the fragmented DNA and apoptotic morphological changes. Points with error bar represents mean±SEM.

FIG. 6: Effect of SNWE and paclitaxel on mRNA expression level in MCF-7 and MDA-MB-231 cells. The total cellular mRNA was isolated and reverse transcribed. The mRNA levels of gene expression in the apoptotic pathway were detected by the BIORAD CFX-96 instrument following the manufacturer's instructions. The genes detected were normalized using GAPDH as a reference gene. The cluster gram heatmap analysis was carried out by MORPHEUS online tool (Morpheus, n.d.).

FIG. 7: Effect of SNWE treatment on apoptotic markers in the MCF-7 and MDA-MB-231 cells. The protein expressions was analysed by immunofluorescence method, the A) BAX, B) Caspase3, and C) p53, expression were increased in the SNWE treatment which shows the induction of caspase dependent apoptosis in breast cancer cells. The cells were immunostained with anti p53, BAX, Caspase3 antibodies and FITC labelled secondary antibodies. DAPI was used as counter stain for nucleus and the images were acquired with fluorescence microscope.

### DETAILED DESCRIPTION OF THE INVENTION

The embodiments herein and the various features and advantageous details thereof are explained more comprehensively with reference to the non-limiting embodiments that are detailed in the following description. Descriptions of well-known components and processing techniques are omitted so as to not unnecessarily obscure the embodiments herein. The examples used herein are intended merely to facilitate an understanding of the ways in which the embodiments herein may be practiced and to further enable those of skill in the art to practice the embodiments herein. Accordingly, the examples should not be construed as limiting the scope of the embodiments herein.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

As used in the description herein, the meaning of “a,” “an,” and “the” includes plural reference unless the context clearly dictates otherwise. Also, as used in the description herein, the meaning of “in” includes “in” and “on” unless the context clearly dictates otherwise.

As used herein, the terms “comprise”, “comprises”, “comprising”, “include”, “includes”, and “including” are meant to be non-limiting, i.e., other steps and other ingredients which do not affect the end of result can be added. The above terms encompass the terms “consisting of” and “consisting essentially of”.

As used herein, the terms “composition” “blend,” or “mixture” are all intended to be used interchangeably.

The present disclosure generally relates to herbal compositions. Specifically, the present disclosure relates to herbal composition comprising of Solanum nigrum along with a pharmaceutically acceptable excipients. The present invention also relates to a method of preparing the herbal composition comprising Solanum nigrum and its anti-proliferative effect.

In an embodiment, the present invention provides a herbal composition comprising a therapeutically effective amount of an extract of Solanum nigrum and one or more pharmaceutically acceptable excipients.

In an embodiment of the present invention, the extract of Solanum nigrum is present in an amount ranging from about 1% to 99% by weight of the composition. Preferably, in an amount ranging from 10% to 99%, 20% to 99%, 30% to 99%, 40% to 99%, 50% to 99%, 60% to 99%, 70% to 99% and 80% to 99% by weight of the composition.

In another embodiment of the present invention, the herbal composition can be used as adjuvants in anti-cancer therapy.

In yet another embodiment of the present invention, the herbal composition is administered in combination with anticancer agents.

In another aspect of the present invention, the anticancer agent is selected from immunomodulating agents, anti-proliferative agents and chemotherapeutic agents.

In an embodiment of the present invention, the immunomodulating agents are compound that stimulates or suppresses the immune system and may help the body fight cancer, infection, or other diseases. Specific immunomodulating agents, such as monoclonal antibodies, cytokines, and vaccines, affect specific parts of the immune system. Examples of immunomodulating agents include but not limited to thalidomide (Thalomid), lenalidomide (Revlimid), and pomalidomide (Pomalyst).

In another embodiment of the present invention, the chemotherapeutic agents such as, erlotinib, bortezomib, disulfiram, epigallocatechin gallate, cyclophosphamide, cytarabine, salinosporamide A, carfilzomib, 17-AAG (geldanamycin), radicicol, lactate dehydrogenase A (LDH-A), fulvestrant, sunitinib, letrozole, imatinib mesylate, oxaliplatin, 5-fluorouracil, rapamycin, lapatinib, lonafarnib, sorafenib, gefitinib, anti-metabolites such as methotrexate; pemetrexed, taxoids, e.g., paclitaxel, ABRAXANE® (Cremophor-free), albumin-engineered nanoparticle formulations of paclitaxel (American Pharmaceutical Partners, Schaumberg, IL.) and docetaxel/doxetaxel; chlorambucil; gemcitabine; 6-thioguanine; mercaptopurine; platinum analogs such as cisplatin and carboplatin; retinoids such as retinoic acid; daunorubicin or doxorubicin; and pharmaceutically acceptable salts, acids and derivatives of any of the above.

In another embodiment of the present invention, the invented herbal composition that can be combined with, but are not limited to, ibrutinib, Fostamatinib, Entospletinib, Cerdulatinib, ruboxistaurin, Tamoxifen, Apitolisib, Gedatolisib, Buparlisib, Copanlisib, Duvelisib, Pictilisib, Taselisib, Oblimersen, navitoclax, venetoclax, baricitinib, tofacitinib, upadacitinib, rituximab blinatumomab, and daratumumab.

In an embodiment, the composition is formulated into any of a solid composition, a semi-solid composition and a liquid composition. In an embodiment, the composition is formulated into a single unit oral dosage form. In an embodiment, the composition is formulated into a topical formulation. In an embodiment, the composition exhibits anti-proliferative activity against breast cancer.

In an embodiment, the herbal composition for treating cancer cells include, optionally, any or a combination of one or more pharmaceutically acceptable excipients. Non-limiting examples of suitable excipients includes a binder, a filler, a non-effervescent disintegrant, an effervescent disintegrant, a preservative, a diluent, a flavoring agent, a sweetener, a lubricant, a dispersing agent, an emulsifier, a coloring agent, a taste masking agent, a pH modifier, a stabilizer, a compaction agent, and combinations of any of these agents. However, a person skilled in the art would appreciate that any other excipient(s), as known to or appreciated by a person skilled in the art can be utilized to serve its intended purpose in the instant invention, without departing from the scope and the spirit of the present disclosure.

In an embodiment, the excipient can be a binder. Non-limiting example of suitable binders include, starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, peptides, and combinations thereof. However, a person skilled in the art would appreciate that any other binder(s), as known to or appreciated by a person skilled in the art can be utilized to serve its intended purpose in the instant invention, without departing from the scope and the spirit of the present disclosure.

In an embodiment, the excipient can be a filler. Non-limiting example of suitable fillers include, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol and combinations thereof. However, a person skilled in the art would appreciate that any other filler(s), as known to or appreciated by a person skilled in the art can be utilized to serve its intended purpose in the instant invention, without departing from the scope and the spirit of the present disclosure.

In an embodiment, the excipient can be a non-effervescent disintegrant. Non-limiting example of suitable non-effervescent disintegrants include, starches (such as corn starch, potato starch, and the like), pregelatinized and modified starches thereof, sweeteners, clays (such as bentonite), micro-crystalline cellulose, alginates, sodium starch glycolate, and gums (such as agar, guar, locust bean, karaya, pecitin, and tragacanth) and combinations thereof. However, a person skilled in the art would appreciate that any other non-effervescent disintegrant(s), as known to or appreciated by a person skilled in the art can be utilized to serve its intended purpose in the instant invention, without departing from the scope and the spirit of the present disclosure.

In an embodiment, the excipient can be an effervescent disintegrant. Non-limiting example of suitable effervescent disintegrants include, sodium bicarbonate in combination with citric acid, and sodium bicarbonate in combination with tartaric acid. However, a person skilled in the art would appreciate that any other effervescent disintegrant(s), as known to or appreciated by a person skilled in the art can be utilized to serve its intended purpose in the instant invention, without departing from the scope and the spirit of the present disclosure.

In an embodiment, the excipient can be a preservative. Non-limiting example of suitable preservatives include, antioxidants (such as alpha-tocopherol or ascorbate) and antimicrobials (such as parabens, chlorobutanol or phenol). In other embodiments, an antioxidant such as butylated hydroxytoluene (BHT) or butylated hydroxyanisole (BHA) may be utilized. However, a person skilled in the art would appreciate that any other preservative(s), as known to or appreciated by a person skilled in the art can be utilized to serve its intended purpose in the instant invention, without departing from the scope and the spirit of the present disclosure.

In an embodiment, the excipient can be a diluent. Non-limiting example of suitable diluents includes pharmaceutically acceptable saccharides, such as, sucrose, dextrose, lactose, microcrystalline cellulose, fructose, xylitol, and sorbitol; polyhydric alcohols; starches; pre-manufactured direct compression diluents; and combination thereof. However, a person skilled in the art would appreciate that any other diluent(s), as known to or appreciated by a person skilled in the art can be utilized to serve its intended purpose in the instant invention, without departing from the scope and the spirit of the present disclosure.

In an embodiment, the excipient can be a flavoring agent. Non-limiting example of suitable flavoring agents include, synthetic flavor oils and flavoring aromatics and/or natural oils, extracts from plants, leaves, flowers, fruits, and combinations thereof. By way of example, these may include cinnamon oils, oil of wintergreen, peppermint oils, clover oil, hay oil, anise oil, eucalyptus, vanilla, citrus oils (such as lemon oil, orange oil, grape and grapefruit oil), and fruit essences (such as apple, peach, pear, strawberry, raspberry, cherry, plum, pineapple, and apricot) and combination thereof. However, a person skilled in the art would appreciate that any other flavoring agent(s), as known to or appreciated by a person skilled in the art can be utilized to serve its intended purpose in the instant invention, without departing from the scope and the spirit of the present disclosure.

In an embodiment, the excipient can be a sweetener. Non-limiting example of suitable sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; stevia-derived sweeteners; chloro derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof. However, a person skilled in the art would appreciate that any other sweetener(s), as known to or appreciated by a person skilled in the art can be utilized to serve its intended purpose in the instant invention, without departing from the scope and the spirit of the present disclosure.

In an embodiment, the excipient can be a lubricant. Non-limiting example of suitable lubricants include magnesium stearate, calcium stearate, zinc stearate, hydrogenated vegetable oils, sterotex, polyoxyethylene monostearate, talc, polyethylene glycol, sodium benzoate, sodium lauryl sulfate, magnesium lauryl sulfate, and light mineral oil and combination thereof. However, a person skilled in the art would appreciate that any other lubricant(s), as known to or appreciated by a person skilled in the art can be utilized to serve its intended purpose in the instant invention, without departing from the scope and the spirit of the present disclosure.

In an embodiment, the excipient can be a dispersants. Non-limiting example of suitable dispersants include starch, alginic acid, polyvinylpyrrolidones, guar gum, kaolin, bentonite, purified wood cellulose, sodium starch glycolate, isoamorphous silicate, and microcrystalline cellulose. However, a person skilled in the art would appreciate that any other dispersant(s), as known to or appreciated by a person skilled in the art can be utilized to serve its intended purpose in the instant invention, without departing from the scope and the spirit of the present disclosure.

In an embodiment, the excipient can be an emulsifier. Non-limiting example of suitable emulsifiers include emulsifying wax, cetearyl alcohol, polysorbate 20, and ceteareth 20, Sodium stearoyl lactylate, Sodium phosphates, soy lecithin and the likes. However, a person skilled in the art would appreciate that any other emulsifier(s), as known to or appreciated by a person skilled in the art can be utilized to serve its intended purpose in the instant invention, without departing from the scope and the spirit of the present disclosure.

In an embodiment, the pharmaceutical composition can include a coloring agent. Non-limiting example of suitable coloring agents include food, drug and cosmetic colors (FD&C), drug and cosmetic colors (D&C), or external drug and cosmetic colors (Ext. D&C). These colors or dyes, along with their corresponding lakes, and certain natural and derived colorants may be suitable for use in the present invention depending on the embodiment. However, a person skilled in the art would appreciate that any other coloring agent(s), as known to or appreciated by a person skilled in the art can be utilized to serve its intended purpose in the instant invention, without departing from the scope and the spirit of the present disclosure.

In an embodiment, the excipient can be a taste-masking agent. Non-limiting example of suitable taste-masking materials include, cellulose hydroxypropyl ethers (HPC), low-substituted hydroxypropyl ethers (L-HPC), cellulose hydroxypropyl methyl ethers (HPMC), methylcellulose polymers and mixtures thereof polyvinyl alcohol (PVA), hydroxyethylcellulose, carboxymethylcelluloses and salts thereof, polyvinyl alcohol and polyethylene glycol co-polymers, monoglycerides or triglycerides, polyethylene glycols, acrylic polymers, mixtures of acrylic polymers with cellulose ethers, cellulose acetate phthalate, and combinations thereof. However, a person skilled in the art would appreciate that any other taste-masking agent(s), as known to or appreciated by a person skilled in the art can be utilized to serve its intended purpose in the instant invention, without departing from the scope and the spirit of the present disclosure.

In an embodiment, the excipient can be a pH modifier. Non-limiting example of suitable pH modifier include sodium carbonate or sodium bicarbonate. However, a person skilled in the art would appreciate that any other pH modifier(s), as known to or appreciated by a person skilled in the art can be utilized to serve its intended purpose in the instant invention, without departing from the scope and the spirit of the present disclosure.

In an embodiment, the weight fraction of the excipient or combination of excipients in the pharmaceutical composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the herbal composition.

The herbal compositions detailed herein can be manufactured in one or several dosage forms. In an embodiment, the dosage form is selected from any or a combination of tablets, including suspension tablets, chewable tablets, effervescent tablets or caplets; pills; powders such as a sterile packaged powder, a dispensable powder, and an effervescent powder; capsules including both soft or hard gelatin capsules such as HPMC capsules, lozenges, a sachet, a sprinkle, a reconstitutable powder or shake, a troche, pellets such as sublingual or buccal pellets, granules, liquids for oral or parenteral administration, suspensions, emulsions, semisolids, or gels. However, any or a combination of pharmaceutical dosage form(s), as known to or appreciated by a person skilled in the art, can be utilized to serve its intended purpose, as laid in the present disclosure, without departing from the scope and spirit of the present invention.

In an embodiment of the present invention, the water extract of S. nigrum treatment induces ROS levels, alters mitochondrial membrane potential, and subsequently induces apoptotic cell death in MCF-7 and MDA-MB-231 cells. The alteration of mitochondrial membrane potential has been considered the initial event of apoptotic cell death in cancer cells. Alterations in mitochondrial membrane potential resulted in the release of pro-apoptotic proteins, which induced apoptotic cell death.

In another embodiment of the present invention, the water extract of S. nigrum treatment alters the mitochondrial membrane potential in a concentration-dependent manner.

In another embodiment, the water extract of S. nigrum of the present invention decreased the accumulation of Rh-123 in treated breast cancer cells.

In another embodiment, the water extract of S. nigrum of the present invention induces apoptotic cell death through ROS formation and subsequent alterations in mitochondrial membrane potential in breast cancer cells.

In another embodiment, the water extract of S. nigrum of the present invention upregulates the pro-apoptotic genes like BAX, P53, BAD, BAM, CASP3, and CASP9 in SNWE treated breast cancer cells which clearly indicated the apoptosis-inducing property of SNWE in breast cancer cells.

In yet another embodiment of the present invention, the water extract of S. nigrum treatment attenuated mRNA expressions of anti-apoptotic markers such as BCL-XL, BCL2, and BCL-W in the breast cancer cell lines.

According to the present invention, the water extract of S. nigrum (SNWE) treatment shows better anti-proliferative and apoptosis-inducing properties in MCF-7 cells than in MDA-MB-231 cells. Although both the cell lines were from the epithelial origin, they were different in their genetic make-up. It has been known that the MCF-7 is an estrogen receptor-positive breast cancer cell line, and it responds well to conventional anticancer therapeutic agents. In contrast, the MDA-MB-231 is a triple-negative breast cancer cell line that is hard to treat by conventional anticancer treatments. Our present results were suggested that SNWE treatment can triggers caspase dependent apoptotic cell death in MCF-7 and MDA-MB-231 cells and further in vivo studies need to be carried out to investigate the anti tumor effects of SNWE.

In an embodiment, the present invention relates to a use of a herbal composition comprising S. nigrum for the manufacture of a medicament for treating a disease or condition, e.g., cancer.

In another embodiment of the present invention, the cancer selected from breast cancer, prostate cancer, brain cancer, colorectal cancer, pancreatic cancer, ovarian cancer, lung cancer, cervical cancer, liver cancer, head/neck/throat cancer, skin cancer, bladder cancer and a hematologic cancer.

In an embodiment, the present invention relates to a use of a herbal composition comprising S. nigrum for the treatment of breast cancer.

The present disclosure may be more fully understood by reference to the following examples:

### EXAMPLES

Preparation of Water Extract of S. nigrum L.

The S. nigrum L. plants were collected from the South Indian regions and air-dried for 7 days to remove the moisture content. The leaves of S. nigrum L. were powdered, and 50 g of powdered plant material was immersed in 250 ml of distilled water. The mixture was taken in the 500 ml conical flask, plugged with sterile cotton, and then kept in a boiling water bath for 60 minutes. This solution was further kept in a shaking incubator for 24 hours and the solution was filtered three times. Finally, a clear water extract of S. nigrum (SNWE) was obtained and stored in the refrigerator at −20° C. for further use. Then the required amount of S. nigrum L water extract (SNWE) was weighed and dissolved in DMSO.

Cell Lines Maintenance and Growth Conditions

Breast carcinoma MDA-MB-231 (Triple-negative) cell lines and MCF-7 (Luminal A) cell lines were procured from NCCS (National Centre for Cell Science, Pune, India). The cells were maintained in DMEM medium supplemented with 10% fetal bovine serum, 50 units/ml penicillin-streptomycin at 37° C. in a 5% CO₂ incubator.

Cytotoxicity Assay

The MCF-7 and MDA-MB-231 cells were seeded in a 96 well plate at a density of 10,000 cells/well and incubated at 37° C. in 5% CO₂ atmospheric condition. Then, cells were treated with SNWE at the maximum concentration of 100 ng/ml. After 72 h of incubation, 100 μl of MTT solution (1 μg/ml) was added to each culture well. The color was allowed to develop for additional 4 h incubation. Then, 100 μl of DMSO was added to dissolve the formazan crystals.

A microplate reader determined the absorbance at 570 nm (Molecular Devices, USA). Percentage cell viability was calculated, and cell-survival curves were constructed.

Intracellular ATP Measurement

The intracellular ATP measurement is the most sensitive method to detect the cell viability. The breast cancer MCF-7 and MDA-MB-231 cells were seeded in a 96 well plates and allowed to reach confluency, then different concentrations of SNWE treatment were given to the cells. The cellular ATP levels to determine the cell viability under the treatment of SNWE for 24 h, 48 h, and 72 h was analysed using a colorimetric ATP assay kit, Sigma-Aldrich, according to the manufacturer instructions. To calculate the unknown concentrations of test values, a standard curve was plotted with the concentration ranges from 0-12 nmole and analyzed by GraphPad Prism 9.0 software. The final concentration of ATP was calculated by the following formula

C=Sa/Sv
where,
C=Concentration of ATP in sample (nmole/μL)
Sₐ=Amount of ATP in unknown sample well (nmole) from standard curve
Sᵥ=Sample volume (μL) added into the well.

Intracellular ROS Measurement

The fluorescent probe 2, 7,-diacetyl dichlorofluorescein diacetate (DCFH-DA) was used to detect ROS generation in MDA MB-231 and MCF-7 cells (Spagnuolo et al., 2006). In brief, the MDA-MB-231 and MCF-7 cells (1×10⁶ cells per well) were seeded in 6 well plates, respectively. After the 72 h treatment, experimental cells were stained with DCFH-DA (1 μg/ml) for 30 minutes under dark conditions. Then, the cells were washed 3 times at 5 minutes intervals with PBS to remove excessive staining. Images were acquired with the fluorescence microscope (Floid Cell Imaging Station, Life Technologies, USA), and the fluorescence intensity measurements were taken excitation at 485±10 and emission at 530±12.5 nm, using a multimode plate reader (Molecular Devices, USA).

Determination of Mitochondrial Membrane Potential

Mitochondrial membrane potential was analyzed using Rhodamine-123 fluorescent staining. The MCF-7 and MDA-MB-231 cells (1×10⁶ cells per well) were seeded in 6 well plates, respectively. After 72 h treatment with SNWE, the cells were stained with Rhodamine-123 for 30 minutes. Then, the cells were washed with PBS thrice to remove excessive staining. Fluorescent measurements were taken, excitation at 450±10 nm and emission at 490±10 nm, using a multimode plate reader (Molecular Devices, USA). Images were acquired with a fluorescence microscope (Floid Cell Imaging Station, Life Technologies, USA).

Apoptosis Assay by Acridine Orange-Ethidium Bromide (AO/EtBr) Dual Staining

Acridine orange/ethidium bromide (AO/EtBr) double staining assay was used for apoptosis detection by morphological observation (Liu et al., 2015). The MCF-7 and MDA-MB-231 cells (1×10⁶ cells per well) were seeded in 6 well plates, respectively. After 72 hours of treatment, both the cells were stained with AO/EtBr in the 1:1 ratio for 30 minutes. Then, the cells were washed with PBS thrice to remove excessive staining. Images were acquired with a fluorescence microscope (Floid Cell Imaging Station, Life Technologies, USA).

Gene Expression Analysis

The qRT-PCR analysis is a highly sensitive and reliable method for gene expression analysis. The total cellular mRNA was isolated using the “Qiagen mini RNAeasy kit” following the manufacturer's instructions, and mRNA levels were quantified by Nano-drop Spectrophotometer (Thermo Scientific, USA). The custom-based pathway-focused gene expression profiling was analyzed by real-time RT-PCR array. Ten prominent apoptotic genes expression at the mRNA level in the SNWE treated breast cancer cell lines were analyzed using the CFX-96 BIO-RAD real-time System. The designed PCR plates have apoptosis pathway genes and the wells P11 to P15 contain a housekeeping gene panel to normalize array data (HK1). Wells P16 to P18 contain genomic DNA controls (GDC). Wells P19 to P21 contain replicate reverse-transcription controls (RTC). The fold changes of gene expression were plotted as cluster grams.

Immunofluorescence

The breast cancer cells were seeded onto 12 well plates at a density of 5000 cells per well and then different concentrations of SNWE treatment were given to the cells. After 72 incubations, the cells were fixed with 100% ice cold methanol for 5 mins, permeabilized with 0.5% triton x-100 for 15 mins, blocked with 5% BSA for 60 mins in room temperature and incubated with primary antibodies in 5% BSA for overnight at 4° C. Then the cells were washed thrice with PBS and incubated with FITC-labelled secondary antibody for 2 h in room temperature. Finally, the cells were washed, counter stained with DAPI and subjected to imaging with fluorescence microscope (Floid Cell Imaging Station, Life Technologies, USA).

Statistical Analysis

Statistical analysis was performed using SPSS 16 (SPSS, Inc., Chicago) statistical package. The data are expressed as mean±standard error of the mean (SEM). One-way analysis of variance (ANOVA) followed by Duncan multiple range test (DMRT) comparison method was used to correlate the difference between the variables. Data are considered statistically significant if values are less than 0.05.

Results

Effect of SNWE Against MCF-7 and MDA-MB-231 Cell Viability

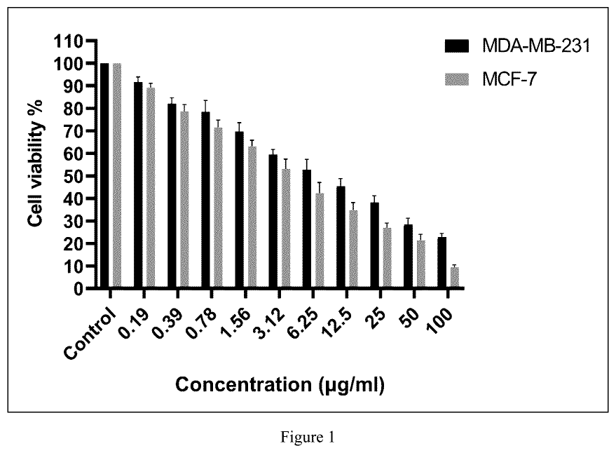

The MCF-7 and MDA-MB-231 cells were treated at different concentrations of SNWE. It was found that SNWE induces cytotoxicity in both the breast cancer cell lines in a concentration dependant manner (FIG. 1). It is also noticed that the anti-proliferative effect of SNWE was more prominent in MCF-7 cell lines than in MDA-MB-231 cells. The IC₅₀ values were found to be 4.26 μg/ml in MCF-7, and 5.3 μg/ml in MDA-MB-231 cells.

Effect of SNWE on Intracellular ATP Levels

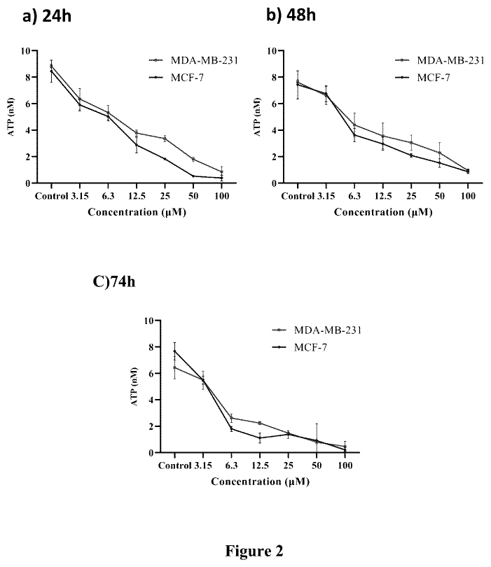

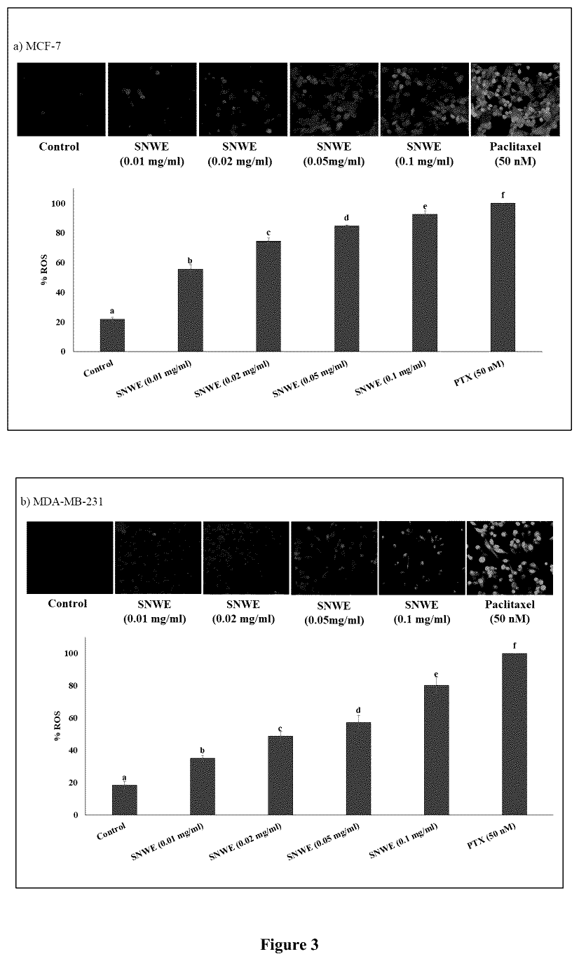

The high energy metabolism rate in cancer cells makes it to produce high amount of ATP. The FIG. 3 shows, the untreated control group have the high ATP concentration and it was significantly reduced by the increasing treatment of SNWE in both the cells. We observed that the cells showed decreased ATP concentration in the concentration dependant manner that directly proportional to the decrease in viability of cells (FIG. 2). The incubation of 24 h, 48 h, and 72 h treatment of 100 μg/ml SNWE showed 0.85±0.07, 0.38±0.1 and 0.20±0.1 nM ATP in MCF-7 cells and 0.94±0.07, 0.84±0.2 and 0.46±0.2 in MDA-MB-231 cells, respectively.

Effect of SNWE on Intracellular ROS Generation in Breast Cancer Cells

The DCFH-DA dye was used to measure the intracellular ROS generation in MCF-7 and MDA-MB-231 cells (FIG. 3). The spectrofluorometric reading showed that SNWE treatment significantly increased intracellular ROS production in both the cell lines. We observed that 0.1 mg/ml of SNWE treatment-induced 92.7±2.4% ROS levels in MCF-7 and 80.4±5.09% ROS in MDA-MB-231 cells, respectively. In addition, the microscopic fluorescence images clearly showed increased DCF-fluorescence in SNWE treated groups compared to the control group.

Effect of SNWE on Mitochondrial Membrane Potential (ΔΨm) in Breast Cancer Cells

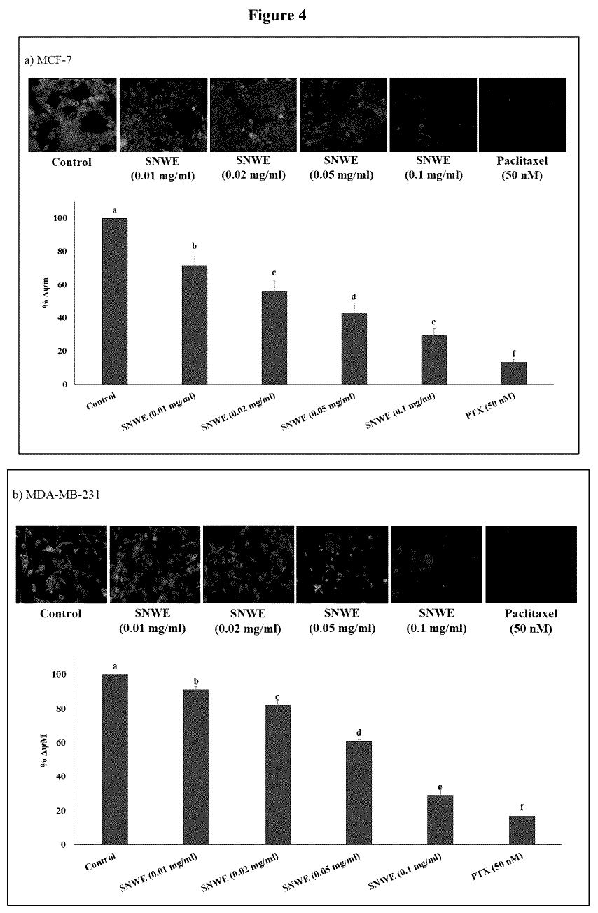

Rhodamine-123 (Rh-123) is a membrane-permeable cationic dye which able to stain the mitochondria in living cells. The SNWE treatment altered the mitochondrial membrane potential (ΔΨm) in a concentration-dependent manner in both the breast cancer cell lines (FIG. 4). We observed that 0.1 mg/ml of SNWE treatment decreased 29.6±4.1% of ΔΨm in MCF-7 and 28.7±4.17% in MDA-MB-231 cells, respectively, when compared to control cells. The non-treated control cells showed high fluorescence due to the accumulation of Rh-123 in the healthy mitochondria (100% ΔΨm).

Effect of SNWE on Apoptotic Morphological Changes in Breast Cancer Cells

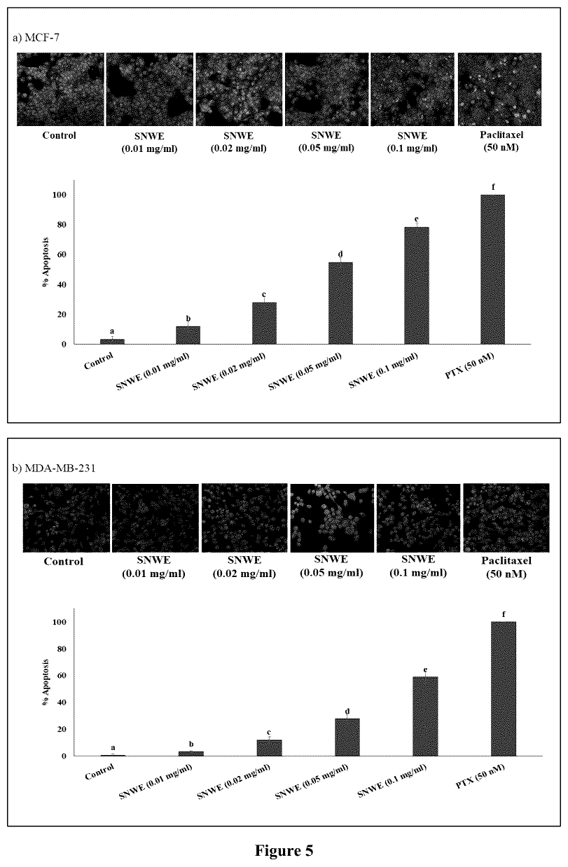

The AO/EtBr dual staining was employed to study the apoptotic morphological changes in cancer cells. We found that 0.1 mg/ml of SNWE significantly increased the apoptotic cellular populations in breast cancer cell lines (FIG. 5). The SNWE treatment-induced 78.4±2.5% of MCF-7 and 58.9±4.2% apoptotic cells in MDA-MB-231 cells, respectively. Conversely, the untreated control cells show no apoptotic cell population. Therefore, the SNWE could induce apoptotic cell death in both MCF-7 and MDA-MB-231 cells.

Effect of SNWE on Gene Expression Analysis

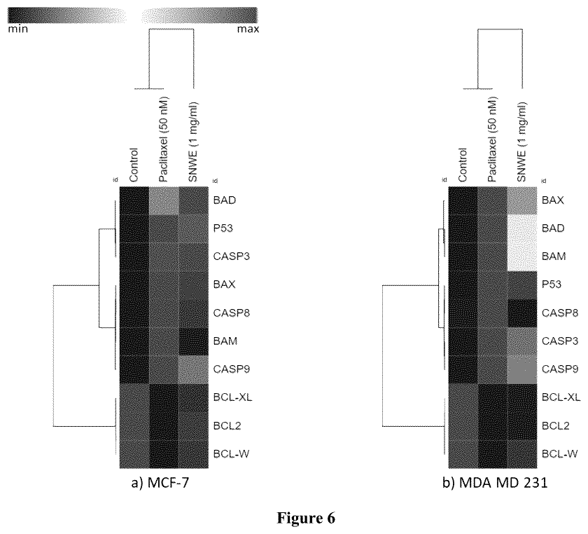

The expression pattern of major apoptotic related genes such as BAX, TP53, BAD, BAM, CASP3, CASP8, CASP9, BCL-XL, BCL2, and BCL-W was analyzed in both MCF-7 and MDA-MB-231 cell lines (FIG. 6). Treatment with SNWE (0.1 mg/ml) induced the mRNA level expressions of proapoptotic genes such as BAX, P53, BAD, BAM, CASP3, CASP8, and CASP9 in MCF-7 and MDA-MB-231 cell lines. Conversely, in both cell lines, the SNWE treatment downregulates mRNA expressions of anti-apoptotic genes like BCL-XL, BCL2, and BCL-W. The fold changes in gene expression data clearly indicate the SNWE treatment higher apoptotic signaling in MCF-7 cell lines than the MDA-MB-231 cell lines.

Effect of SNWE on Apoptotic Markers

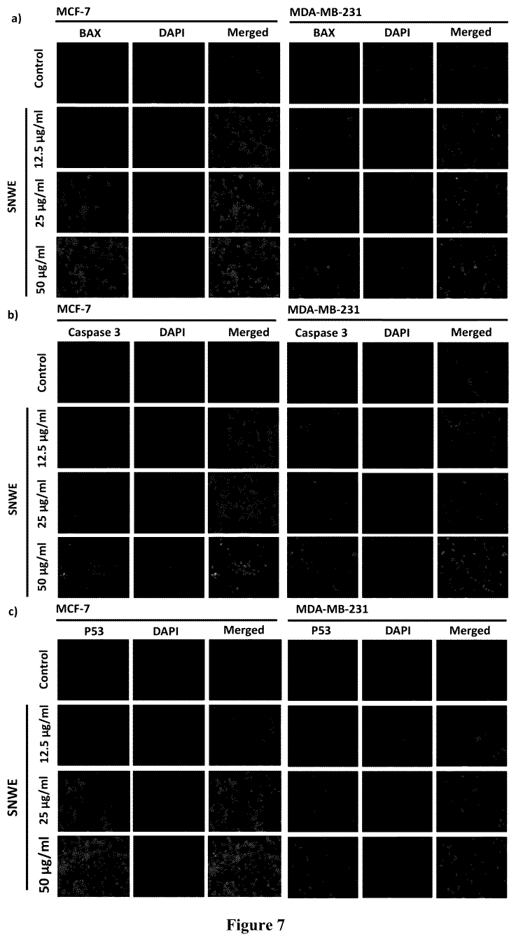

To clarify whether SNWE treatment decreased the cell viability by activation of programmed cells death in MCF-7 and MDA-MB-231 cells, by BAX, p53, caspase 3 was determined by immunofluorescence analysis. The BAX, caspase-3 and p53 is the essential marker for caspase dependant apoptosis. Results showed in FIG. 7, indicated that significant increase of A) BAX, B) Caspase-3 and C) p53 in MCF-7 and MDA-MB-231 cells in concentration dependent treatment of SNWE. These, results suggested that SNWE mainly activating the caspase dependent apoptotic pathway, which might result in the cytotoxicity of two tested breast cancer cells.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

### Advantages of the Invention

The present disclosure provides a herbal composition comprising Solanum nigrum for the effective treatment of cancer.

The present disclosure provides a herbal composition comprising Solanum nigrum for the effective treatment of cancer that is safe without any side effects.

The present disclosure provides a herbal composition comprising Solanum nigrum for the effective treatment of cancer that is cheaper.

### REFERENCES

Sun, Y.-S., Zhao, Z., Yang, Z.-N., Xu, F., Lu, H.-J., Zhu, Z.-Y., Shi, W., Jiang, J., Yao, P.-P., & Zhu, H.-P. (2017). Risk Factors and Preventions of Breast Cancer. International Journal of Biological Sciences, 13(11), 1387. https://doi.org/10.7150/IJBS.21635.
Akram, M., Iqbal, M., Daniyal, M., & Khan, A. U. (2017). Awareness and current knowledge of breast cancer. Biological Research 2017 50:1, 50(1), 1-23. https://doi.org/10.1186/S40659-017-0140-9.
Cortazar, P., Zhang, L., Untch, M., Mehta, K., Costantino, J. P., Wolmark, N., Bonnefoi, H., Cameron, D., Gianni, L., Valagussa, P., Swain, S. M., Prowell, T., Loibl, S., Wickerham, D. L., Bogaerts, J., Baselga, J., Perou, C., Blumenthal, G., Blohmer, J., . . . Von Minckwitz, G. (2014). Pathological complete response and long-term clinical benefit in breast cancer: the CTNeoBC pooled analysis. The Lancet, 384(9938), 164-172. https://doi.org/10.1016/S0140-6736(13)62422-8.
Waks, A. G., & Winer, E. P. (2019). Breast Cancer Treatment: A Review. JAMA, 321(3), 288-300. https://doi.org/10.1001/JAMA.2018.19323.
Carey, L. A., Perou, C. M., Livasy, C. A., Dressler, L. G., Cowan, D., Conway, K., Karaca, G., Troester, M. A., Tse, C. K., Edmiston, S., Deming, S. L., Geradts, J., Cheang, M. C. U., Nielsen, T. O., Moorman, P. G., Earp, H. S., & Millikan, R. C. (2006). Race, Breast Cancer Subtypes, and Survival in the Carolina Breast Cancer Study. JAMA, 295(21), 2492. https://doi.org/10.1001/jama.295.21.2492.
Guilford, J. M., & Pezzuto, J. M. (2008). Natural products as inhibitors of carcinogenesis. Http://Dx.Doi.Org/10.1517/13543784.17.9.1341, 17(9), 1341-1352.
Campisi, A., Acquaviva, R., Raciti, G., Duro, A., Rizzo, M., & Santagati, N. A. (2019). Antioxidant Activities of Solanum nigrum L. Leaf Extracts Determined in In Vitro Cellular Models. Foods 2019, Vol. 8, Page 63, 8(2), 63. https://doi.org/10.3390/FOODS8020063.

1. A herbal composition comprising a therapeutically effective amount of an extract of Solanum nigrum and one or more pharmaceutically acceptable excipients.

2. The herbal composition as claimed in claim 1, wherein the extract of Solanum nigrum is an extract of leaves of Solanum nigrum, an extract of fruits of Solanum nigrum, or an extract of whole plant of Solanum nigrum.

3. The herbal composition as claimed in claim 1, wherein the extract of Solanum nigrum is water extract of Solanum nigrum.

4. The herbal composition as claimed in claim 1, wherein the extract of Solanum nigrum is present in an amount ranging from about 1% to about 99% by weight of the composition.

5. The herbal composition as claimed in claim 1, wherein the composition is administered as adjuvants in cancer therapy.

6. The herbal composition as claimed in claim 1, wherein the composition is administered in combination with anticancer agents.

7. The herbal composition as claimed in claim 5, wherein the anticancer agent is selected from immunomodulating agents, anti-proliferative agents and chemotherapeutic agents.

8. A method of treating a cancer comprising administering a therapeutically active amount of the herbal composition as claimed claim 1 to a subject in need thereof.

9. The method of treating a cancer as claimed in claim 8, wherein the cancer is breast cancer, prostate cancer, brain cancer, colorectal cancer, pancreatic cancer, ovarian cancer, lung cancer, cervical cancer, liver cancer, head/neck/throat cancer, skin cancer, bladder cancer and a hematologic cancer.

10. The method of treating a cancer as claimed in claim 8, wherein the cancer is breast cancer.
